(19) United States
(12) Patent Application Publication
(10) Pub. No.: US 2026/0108667 A1
(43) Pub. Date: Apr. 23, 2026

(54) MEDICAL DEVICES COMPRISING A VENTILATION MECHANISM AND RELATED METHODS AND SYSTEMS

(71) Applicant: Boston Scientific Scimed, Inc., Maple Grove, MN (US)

(72) Inventors: Steven T. CARLSON, Minneapolis, MN (US); John SUBASIC, Sutton, MA (US); Benjamin CLEVELAND, Bellingham, MA (US); Kenneth FLYNN, Woburn, MA (US); Rodolfo FINOCCHI, Ashland, MA (US); Timothy Oyeniran ALADE, Cincinnati, OH (US)

(73) Assignee: Boston Scientific Scimed, Inc., Maple Grove, MN (US)

(21) Appl. No.: 19/361,533

(22) Filed: Oct. 17, 2025

Related U.S. Application Data
(60) Provisional application No. 63/708,899, filed on Oct. 18, 2024.

(51) Int. Cl. A61M 1/00

(52) U.S. Cl. CPC A61M 1/76; A61M 1/7411; A61M 2205/3344; A61M 2205/586

(57) ABSTRACT

Medical devices, systems, and related methods are described. The medical device may include a handle with a hub, a suction port, and a ventilation mechanism; and a sheath integral with or coupled to the handle. The hub may define a lumen and a proximal opening configured to receive a shaft of a medical instrument, and the suction port may be in fluid communication with the lumen and configured to be connected to a vacuum source. The ventilation mechanism may include a housing coupled to the hub and may be movable to cover and uncover at least one aperture of the hub or the suction port.

### CROSS-REFERENCE TO RELATED APPLICATION(S)

This application claims the benefit of priority to U.S. Provisional Application No. 63/708,899, filed on Oct. 18, 2024, which is incorporated by reference herein in its entirety.

### TECHNICAL FIELD

Aspects of the present disclosure relate generally to medical devices such as aspiration devices and related systems and methods. More specifically, the present disclosure relates to medical systems and devices with suction channels and ports, e.g., to facilitate aspiration during medical procedures.

### BACKGROUND

During a medical procedure, medical professionals often use medical devices coupled to a vacuum source to provide suction or irrigation during the medical procedure. However, the vacuum source is typically in an on state or off state, and it would be useful to have enhanced control over a resulting vacuum pressure applied to the medical device during the medical procedure.

### SUMMARY

The present disclosure relates to, among other things, medical devices and systems useful for medical procedures, e.g., for controlling suction. Each aspect disclosed herein may include one or more features described in connection with any other disclosed aspect.

The present disclosure includes, for example, a medical device including a handle and a sheath integral with or coupled to the handle. The handle may include a hub defining a lumen and a proximal opening configured to receive a shaft of a medical instrument, a suction port in fluid communication with the lumen and configured to be connected to a vacuum source, and a ventilation mechanism including a housing coupled to the hub. The sheath may define a channel in fluid communication with the suction port. The hub or the suction port may include at least one aperture. The ventilation mechanism may include an actuator movable relative to the hub to cover and uncover the at least one aperture of the hub or the suction port. The actuator may be an outer surface of the housing or may be movably coupled to the housing. According to some aspects, for example, an outer surface of the housing is the actuator, e.g., the housing being movable relative to the hub. In other aspects, the actuator may be movably coupled to the housing, e.g., the housing being fixed to the hub.

According to some aspects of the present disclosure, the suction port may include the at least one aperture and/or the housing may be pivotably coupled to the hub. The ventilation mechanism may include a spring within the housing, the spring being configured to bias the housing toward the suction port. According to some aspects, the hub may include the at least one aperture. The housing may be fixed to the hub and the actuator may be movably coupled to the housing.

In some examples, the actuator may be slidable along a surface, such as an inclined surface, of the housing. The housing may surround a portion of the hub. The housing may be slidable relative to the hub, e.g., along an axis perpendicular to a longitudinal axis of the hub. The housing may be rotatable relative to the hub, e.g., about the axis perpendicular to the longitudinal axis of the hub.

According to some aspects, the hub may include a tab, a hook, or a flattened portion distal to the housing and configured to be gripped by a hand of a user. The at least one aperture of the hub or the suction port may have a variable cross-sectional profile. The at least one aperture may include a plurality of apertures. For example, the plurality of apertures may include a first aperture adjacent to a second aperture at a same axial position along a length of the hub, or a first aperture proximal to a second aperture along a length of the hub.

The present disclosure also includes a medical device comprising a handle including a hub defining a lumen and a proximal opening configured to receive a shaft of a medical instrument, a suction port in fluid communication with the lumen and configured to be connected to a vacuum source, and a ventilation mechanism including a housing coupled to the hub, wherein the hub includes at least one aperture, and wherein the ventilation mechanism includes an actuator movable relative to the hub to cover and uncover the at least one aperture. The medical device may also include a sheath coupled to the handle and defining a channel in fluid communication with the lumen of the hub. In some examples, the housing surrounds a portion of the hub, the housing being slidable or rotatable relative to the hub. In other examples, the housing is fixed to the hub and the actuator is slidable along a surface of the housing.

The present disclosure also includes a medical device comprising a handle including a hub defining a lumen and a proximal opening configured to receive a shaft of a medical instrument, a suction port in fluid communication with the lumen and configured to be connected to a vacuum source, and a ventilation mechanism including a housing coupled the hub proximate the at least one aperture, wherein the hub includes at least one aperture, and wherein the housing is slidable relative to the hub to cover and uncover the at least one aperture. The medical device may further comprise a sheath coupled to the handle and defining a channel in fluid communication with the lumen of the hub. The hub may include a tab, a hook, or a flattened portion distal to the housing and configured to be gripped by a hand of a user.

### BRIEF DESCRIPTION OF THE FIGURES

The accompanying drawings, which are incorporated in and constitute a part of this application, illustrate aspects of this disclosure and together with the description, serve to explain the principles of this disclosure. The drawings show exemplary medical devices useful for providing and controlling suction during a medical procedure.

FIGS. 1A and 1B depict an exemplary medical device that includes a rotatable ventilation mechanism, according to some aspects of the present disclosure.

FIG. 2 depicts an exemplary medical device that includes a rotatable ventilation mechanism, according to some aspects of the present disclosure.

FIG. 3 depicts another exemplary medical device that includes a rotatable ventilation mechanism, according to some aspects of the present disclosure.

FIGS. 4A and 4B depict another exemplary medical device that includes a rotatable ventilation mechanism, according to some aspects of the present disclosure.

FIG. 5 depicts another exemplary medical device according to some aspects of the present disclosure.

FIGS. 6, 7, 8A-8B, and 9 depict additional exemplary medical device that includes a slidable ventilation mechanism, according to some aspects of the present disclosure, including indicators and/or ergonomic features.

### DETAILED DESCRIPTION

Both the foregoing general description and the following detailed description are exemplary and explanatory only and are not restrictive of the features, as claimed. As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Relative terms such as “about,” “substantially,” and “approximately,” etc., are used to indicate a possible variation of ±10% of the stated numeric value or range. The terms “proximal” and “distal” are used herein to refer to the relative positions of the components of exemplary medical devices. As used herein, “proximal” refers to a position relatively closer to the exterior of the body or closer to an operator using the medical device (see proximal “P” and distal “D” directional arrows in the figures). In contrast, “distal” refers to a position relatively further away from the operator using the medical device, or closer to the interior of the body.

Aspects of the present disclosure are described herein with reference to exemplary medical devices, systems, and methods that may be useful for facilitating suction and/or irrigation during medical procedures. For example, the medical device may be an aspiration device. In some examples, the medical device includes a handle including a hub and a suction port in fluid communication with a lumen of the hub. The handle may include a ventilation mechanism, e.g., useful for controlling vacuum pressure provided via the suction port. The medical device also may include a sheath coupled to the handle and defining a channel in fluid communication with the suction port. For example, the medical device may be configured to receive a medical instrument, e.g., via a proximal opening of the hub. A shaft of the medical instrument may extend through the hub and the sheath, e.g., the medical instrument used to deliver irrigation at or proximate a target site of a subject while the medical device is used to apply suction at or proximate the target site. The medical device may be configured to allow a user to adjust suction pressure with the same hand used to grasp the handle of the medical device. For example, the user may employ the ventilation mechanism having features to cover and uncover the one or more apertures of the suction port and/or the hub. The ventilation mechanism may include an actuator, wherein the actuator is part of the housing (e.g., an outer surface of the housing) or is movably coupled to the housing. When the medical device receives the shaft of a medical instrument, the channel of the sheath may radially surround the shaft of the medical instrument to apply suction, e.g., to remove fluid(s) and/or other materials from the target site of a subject, via the suction port during a medical procedure. The handle may be configured to control suction pressure in the medical device during medical procedures, such that covering and uncovering at least one aperture of the suction port and/or the hub allows the user to adjust and control magnitude of suction pressure therein.

References to a particular type of procedure, such as a urology procedure; a medical device, such as an aspiration device; and/or an organ, such as a kidney, are provided for convenience and not intended to limit this disclosure. Accordingly, analogous medical devices or systems may utilize one or more aspects of exemplary medical devices, systems, and methods described herein. Reference is now made in detail to examples to help illustrate aspects of the present disclosure through the accompanying drawings. Wherever possible, the same reference numbers will be used throughout the drawings to refer to the same or like parts.

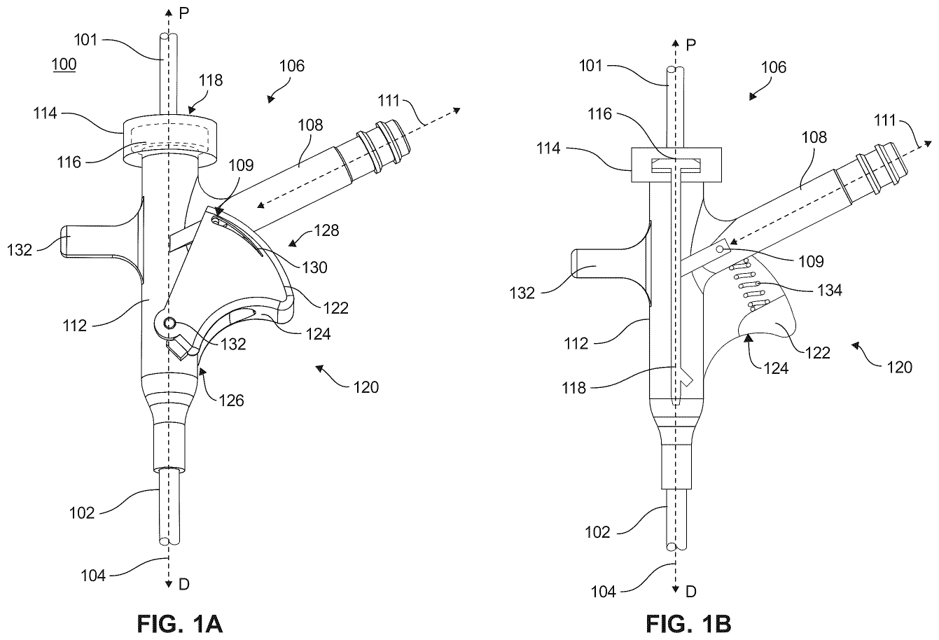

FIGS. 1A-1B illustrate an exemplary medical device 100 according to some aspects of this disclosure, medical device 100 including a handle 106 and a sheath 102. The medical device 100 may be configured to allow a user (e.g., medical professional) to deliver medical instruments to access internal areas of a subject's body, e.g., to perform medical diagnoses and/or treatments on the subject. The handle 106 may be coupled to or integral with the sheath 102, such that the handle 106 is in fluid communication with a channel of the sheath 102. The handle 106 may include a hub 112 with a proximal portion 114 defining a proximal opening 116 to slidably receive a medical instrument 101 such as a scope (e.g., ureteroscope, endoscope, hysteroscope, bronchoscope, cystoscope, or other scope or similar device) into a lumen 118 defined by the handle 106. The medical instrument 101 may be or include a shaft that is slidable along the lumen 118 of the handle 106 and into the channel of the sheath 102. In some examples, the medical instrument 101 may be useful to deliver irrigation to a subject and/or provide tools to a target site of the subject. The proximal opening 116 may include a seal configured to maintain a position of the medical instrument 101 relative to the medical device 100, forming a substantially airtight and/or watertight seal around the medical instrument 101 received therein (e.g., via the proximal opening 116 formed in the proximal portion 114 of the hub 112).

The handle 106 also includes a suction port 108 in fluid communication with the lumen 118 of hub 112. The suction port 108 may be integral with or coupled to the hub 112, e.g., extending along an axis 111 transverse relative to a longitudinal axis 104 of the handle 106. The handle 106 may include one or more features that allow the user to control or adjust pressure through the suction port 108 when the suction port 108 is coupled to a vacuum source during a medical procedure. In the example shown in FIGS. 1A-1B, the handle 106 includes a ventilation mechanism 120 including a housing 122 coupled to the hub 112 proximate an aperture 109 formed in the suction port 108. The ventilation mechanism 120 may be positioned such that a user may be able to grasp the hub 112 and control movement of the housing 122 to cover and uncover the aperture 109, for example using the same hand grasping the hub 112.

In this example, the housing 122 of the ventilation mechanism 120 may be movably coupled to the suction port 108 proximate the aperture 109. As shown, the housing 122 is coupled to the suction port 108 via a hinge 132 of the ventilation mechanism 120, such that the housing 122 may be movable between a plurality of positions. For example, the housing 122 may be movable between a first position wherein the aperture 109 is fully uncovered and a second position wherein the aperture 109 is fully covered by the housing 122, as well as any position therebetween wherein the aperture 109 is partially covered. In the example shown in FIGS. 1A-1B, the housing 122 includes an outer surface 124 having a first end 126 coupled to the hub 112 via the hinge 132, and a second end 128 opposite the first end 126. The housing 122 may pivot about the hinge 132 between the first position and second position to cover and uncover the aperture 109, in addition to any position therebetween partially covering the aperture 109. The housing 122 may define a slot or cavity therein shaped and sized to accommodate the hub 112, the suction port 108, or portions thereof. For example, the housing 122 may surround a portion of the hub 112 and/or a portion of the suction port 108 received via the slot therein. The outer surface 124 may define an actuator of the ventilation mechanism 120 that receives user input, such that actuating or pressing the outer surface 124 in a direction toward the suction port 108 actuates or moves the housing 122 relative to the suction port 108 (e.g., between the first position, the second position, or any position therebetween).

In some aspects, the housing 122 of the ventilation mechanism 120 includes one or more openings formed therethrough. For example, the housing 122 may include an opening 130. As the user rotates the housing 122 relative to the suction port 108, the opening 130 may move over the aperture 109 to provide fluid communication therebetween (e.g., to uncover the aperture 109). In some examples, the opening 130 may have variable dimensions along an extent thereof, for example a variable cross-sectional profile. In some examples, the opening 130 has a cross-sectional profile that increases in a direction toward the suction port 108, and in others the opening 130 has a cross-sectional profile that decreases in the direction toward the suction port 108. The specific cross-sectional profile may influence a rate of change of a magnitude of pressure therethrough, for example such that the outer surface 124 of the housing 122 serving as an actuator of the ventilation mechanism 120 may cause linear change in the magnitude of pressure (e.g., during the medical procedure when the suction port 108 is connected to a vacuum source). Therefore, as the user rotates the housing 122 via the hinge 132 between various positions relative to the suction port 108, this in turn may cause the opening 130 in the housing 122 to slide over and relative to the aperture 109 in the suction port 108.

In some aspects, the ventilation mechanism 120 may include one or more springs (e.g., compression spring, extension spring, torsion spring, etc.), which may be configured to bias the housing 122 relative to the suction port 108. In the cross-sectional view shown in FIG. 1B, the ventilation mechanism 120 includes a spring 134 disposed within the housing 122, the spring 134 being configured to bias the housing 122 relative to the suction port 108. For example, the spring 134 may be a compression spring including a fixed portion secured to the suction port 108 proximate the aperture 109 and a movable portion secured to an inner surface or portion of the housing 122 opposite the fixed portion of spring 134 (e.g., opposite the outer surface 124 of the housing 122). As the user actuates or presses the outer surface 124, the housing 122 may displace the movable portion of spring 134 toward the fixed portion, and thus compress the spring 134. As the user releases outer surface 124, the spring 134 may decompress which in turn may cause the outer surface 124 to move in a direction away from the suction port 108 as the housing 122 pivots between various positions via the hinge 132. In some examples, the spring 134 may bias the housing 122 toward a fully closed position wherein the housing 122 covers the aperture 109, and in other examples, the spring 134 may bias the housing 122 toward a fully open position wherein the housing 122 uncovers the aperture 109. In other implementations, the ventilation mechanism 120 may include one or more extension springs, such that each extension spring includes a fixed end secured to the suction port 108 and a movable end secured to the housing 122 opposite the fixed end. The extension spring(s) may support moving the housing 122 in a direction toward the suction port 108 as the housing 122 pivots between various positions via the hinge 132. The handle 106 may include one or more surface features configured to receive or otherwise couple with respective ends of the springs, for example one or more hooks extending outwardly from the suction port 108 that is configured to engage the fixed end(s) of the extension spring(s).

In some examples, the ventilation mechanism 120 may include a latch, loop, thumb ring, or other feature configured to assist the user with grasping the housing 122. For example, the ventilation mechanism 120 may include a finger loop formed on or coupled to the housing 122, e.g., extending outwardly from the outer surface 124 in a direction away from the suction port 108. This may be useful, for example, to facilitate grasping and moving the housing 122 between various positions relative to the suction port 108 to cover, partially cover or completely ventilate the aperture 109.

Optionally, the ventilation mechanism 120 may include a sealing element coupled to or otherwise disposed on an inner surface of the housing 122 proximate the aperture 109. The sealing element may include a deformable, compressible, or otherwise moldable material configured to provide an airtight or watertight seal against the aperture 109. For example, the sealing element may include an O-ring or a rubber material capable of conforming to the perimeter of the aperture 109. In some examples, as the user moves the housing 122 relative to the suction port 108, the sealing element moves away from, or no longer conforms to, the aperture 109 (e.g., as the opening 130 moves into fluid communication with the aperture 109).

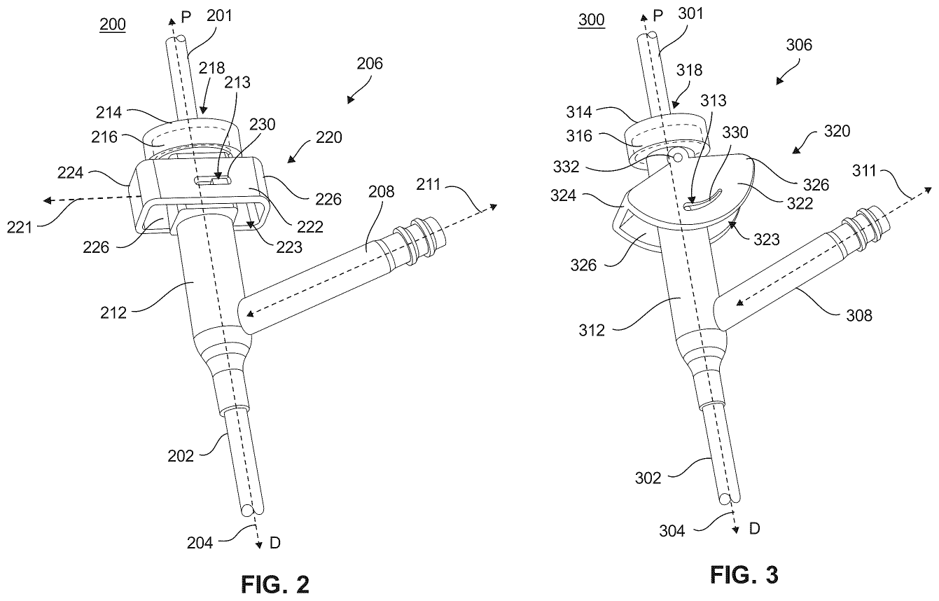

FIG. 2 illustrates another exemplary medical device 200 according to aspects of the disclosure. Unless otherwise specified, the medical device 200 may include any of the features of the medical device 100. As shown, the medical device 200 includes a handle 206 and a sheath 202 defining a channel in fluid communication with a lumen of the handle 206. The handle 206 includes a hub 212 defining the lumen 218, and the hub 212 including a proximal portion 214 that defines a proximal opening 216 in communication with the lumen 218. The handle 206 includes a suction port 208 in fluid communication with the lumen 218 of the hub 212, e.g., the suction port 208 extending along an axis 211 transverse to a longitudinal axis 204 of the hub 212 and configured to be connected to a vacuum source.

The handle 206 also includes a ventilation mechanism 220 proximate one or more apertures of the hub 212, the aperture(s) being in fluid communication with the lumen 218. In this example, the ventilation mechanism 220 includes a housing 222 that is movable (e.g., slidable) along an axis 221 approximately perpendicular to the longitudinal axis 204. Thus, for example, an outer surface of the housing 222 may serve as an actuator of the ventilation mechanism 220, e.g., receiving user input. The housing 222 defines a cavity 223 therein shaped and sized to receive the hub 212 therein. The user may slide the housing 222 along the axis 221 relative to an aperture 213 formed in the hub 212, for example to control pressure within the medical device 200 when the suction port 208 is fluidly coupled to a vacuum source. The user may slide the housing 222 along the axis 221 to cover and uncover the aperture 213 of the hub 212, and thereby control pressure in the handle 206. In at least one example, the user may slide a first end 224 of the housing 222 in a first direction along the axis 221 to cover the aperture 213 (e.g., seal or close the aperture 213), and may slide a second end 226 of the housing 222 in a second direction opposite the first direction along the axis 221 to uncover the aperture 213 (e.g., ventilate or open the aperture 213). Thus, for example, the user may use one hand to control the medical device 200 (and adjust pressure therein) and the other hand to insert a shaft 201 of a medical instrument (e.g., a scope) through the proximal opening 216 into the lumen 218.

In some aspects, the housing 222 of the ventilation mechanism 220 may be movable between various positions relative to the hub 212 to increase or decrease pressure during the medical procedure. The housing 222 may include one or more openings that can align with the aperture 213. In the example shown in FIG. 2, the housing 222 includes an opening 230 slidable relative to the aperture 213 by sliding the housing 222 to cover and uncover the aperture 213. Thus, for example, this sliding movement may cause an increase or decrease of pressure, e.g., based on alignment of the opening 230 with the aperture 213.

In some examples, the opening 230 may have a variable cross-sectional profile, such as a variable width of the opening 230 along an extent of the housing 222. For instance, the opening 230 may have a cross-sectional width that increases along the axis 221 in either the first direction and/or the second direction. The housing 222 may have a plurality of openings in fluid communication with the cavity 223 therein, for example openings similar to or the same as the opening 230 shown in FIG. 2. Further, the opening 230 (or plurality of openings) may have different sizes and/or different shapes (e.g., circular shape, teardrop shape, triangle shape, etc.). It should also be understood that the hub 212 may have a plurality of apertures in fluid communication with the lumen 218, for example apertures similar to or the same as the aperture 213 shown in FIG. 2 to support fluid communication with the opening 230 in the housing 222 (or plurality of openings formed therein).

FIG. 3 illustrates another exemplary medical device 300, which may include any of the features of the medical device 100 and/or medical device 200 unless otherwise specified. As shown, the medical device 300 includes a handle 306, and a sheath 302 defining a channel in fluid communication with a lumen of the handle 306. The handle 306 includes a hub 312 defining the lumen 318 therein, for example in fluid communication with the channel of the sheath 302. The hub 312 includes a proximal portion 314 including a proximal opening 316 capable of receiving a shaft 301 or the like of a medical instrument therein. The hub 312 also includes one or more apertures 313 in fluid communication with the lumen 318 therein. The handle 306 also includes a suction port 308 in fluid communication with the lumen 318 of the hub 312, e.g., the suction port 308 extending along an axis 311 transverse to a longitudinal axis 304 of the medical device 300.

In some aspects, the handle 306 includes a ventilation mechanism 320 coupled to the hub 312 proximate the aperture(s) 313. In this example, the ventilation mechanism 320 includes a housing 322 defining a cavity 323 therein and an opening 330 extending therethrough. The housing 322 may be pivotably coupled to the hub 312, e.g., via a hinge 332 of the ventilation mechanism 320, such that a user may grasp and move a first end 324 or a second end 326 of the housing 322 relative to the hub 312. Thus, for example, the housing 322 may serve as an actuator of the ventilation mechanism 320. In these examples, the housing 322 may rotate between various positions, e.g., a first position in which the housing 322 fully covers the aperture 313 in the hub 312 (e.g., closed position) and a second position in which the housing 322 fully uncovers the aperture 313 (e.g., open position). As the user moves the housing 322 relative to the hub 312 from the first position toward the second position, the opening 330 in the housing 322 gradually moves in alignment with the aperture 313 in the hub 312. A rate of change in pressure may be customizable, for example based on the size, shape, and/or position of the opening 330 of the housing 322 relative to the size, shape, and/or position of the aperture 313 of the hub 312.

The handle 306 may also include one or more retention features configured to retain the housing 322 in at least one of the closed position or the open position. For example, the handle 306 may include snap-fit or compression fit features configured to retain engagement between the hub 312 and the housing 322. In some examples, the outer surface of the hub 312 may define a groove or slot configured to movably receive the housing 322 therein, such that the groove or slot defines one or more movement boundaries as the housing 322 moves relative to the hub 312. The groove or slot may be configured to contact or otherwise engage the housing 322 while in the first position and/or the second position, for example to define a first boundary corresponding with the first position of the housing 322 and a second boundary corresponding with the second position of the housing 322. In some examples, one or more inner surfaces of the housing 322 may be shaped and sized to contact or otherwise engage portions of the outer surface of the hub 312. For example, moving the ventilation mechanism 320 from the first position toward the second position may cause the inner surface of the first end 324 or the second end 326 of the housing 322 to move into contact or engagement with the outer surface of the hub 312, thus defining a movement boundary for the housing 322 during the medical procedure to inhibit further movement relative to the hub 312.

In some implementations, the ventilation mechanism 320 may include a biasing mechanism similar to that of medical device 200, e.g., configured to bias the housing 322 relative to the hub 312.

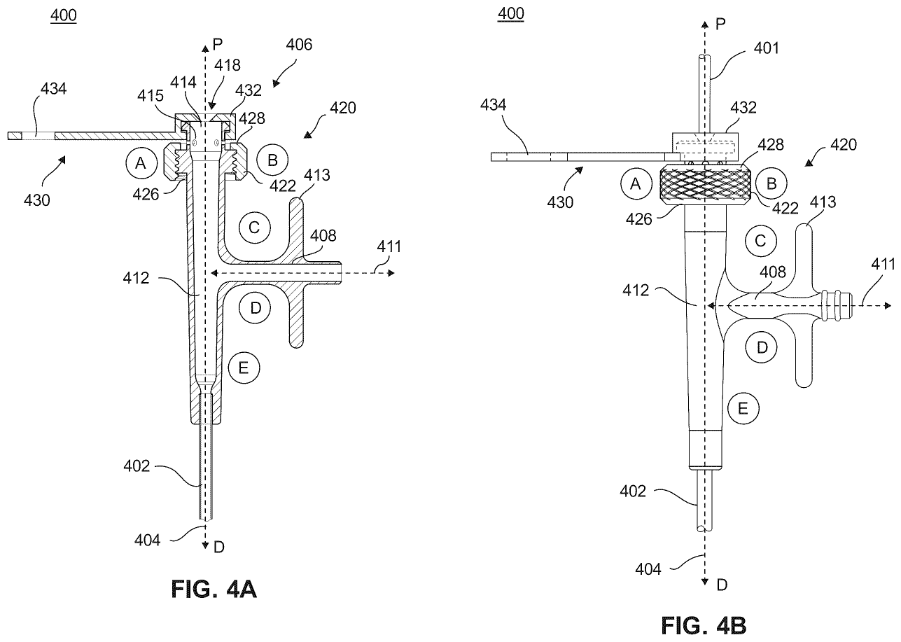

FIGS. 4A-4B illustrate another exemplary medical device 400 according to aspects of the disclosure. Unless otherwise specified, the medical device 400 may include any of the features of the medical devices 100, 200 and/or 300. The medical device 400 includes a handle 406 and a sheath 402. The handle 406 includes a hub 412 defining a lumen 418 in fluid communication with a channel of the sheath 402. The handle 406 also includes a suction port 408 extending along an axis 411, the suction port 408 configured to be connected to a vacuum source, e.g., to provide suction pressure through the medical device 400 during a medical procedure. The lumen 418 extends along an axis 404 transverse to the axis 411 of the suction port 408. The hub 412 includes a proximal portion 414 defining a proximal opening in communication with the lumen 418, for example shaped and sized to receive a shaft 401 of a medical instrument therein.

The hub 412 also includes a plurality of apertures 415, e.g., three apertures, in fluid communication with the lumen 418. In other examples, the plurality of apertures 415 may include two, four, or five or more apertures 415. The apertures 415 may have same size and/or shape, or may have different sizes and/or shapes from each other (e.g., apertures of same shape but different sizes, different shapes and the same sizes, etc.). In some examples, the apertures 415 may be arranged along the proximal portion 414 to increase or decrease in size in a direction towards the suction port 408. Further, for example, the apertures 415 may be arranged at the same axial position or adjacent to each other along the longitudinal axis 404 of the hub 412. For example, the apertures 415 may include a first aperture adjacent to a second aperture at a same axial position along a length of the hub 412 (as illustrated in FIGS. 4A-4B) and/or may include an aperture proximal to another aperture.

The handle 406 may also include a ventilation mechanism 420 coupled to the hub 412 proximate the apertures 415. The ventilation mechanism 420 includes a housing 422 that is rotatable about the hub 412 to cover and uncover the apertures 415. Thus, for example, the housing 422 may serve as an actuator of the ventilation mechanism 420. The housing 422 includes a distal surface 426 and a proximal surface 428 defining a cavity therein, e.g., the cavity being shaped and sized to receive the hub 412 therethrough. As shown in the partial cross-section view in FIG. 4A, the housing 422 and the hub 412 may include complementary threaded portions. The user may rotate the housing 422 relative to the hub 412 to gradually cover or uncover the apertures 415 to control pressure of medical device 400. The user may grasp the handle 406 with the same hand used to rotate the housing 422.

In some examples, the handle 406 may include one or more surface features, such as features on the suction port 408 to facilitate ergonomic grasping by the user. For example, the handle 406 may include a protrusion 413 extending outwardly from the outer surface of the suction port 408 relative to the axis 411. The protrusion 413 may include a flattened surface or portion distal to the housing 422 relative to the axis 411, for example the protrusion 413 configured to be gripped by the hand of the user distal to the housing 422. The protrusion 413 may include a first portion and a second portion that are positioned diametrically opposite with respect to the suction port 408 and the axis 411, such that the protrusion 413 extends radially outward from the suction port 408 relative to the axis 411 in at least two directions (e.g., opposite directions away from the suction port 408). The protrusion 413 may be useful, for example, to support the user's hand to grasp and control the suction port 408.

In some examples, the medical device 400 may include a tether coupled to the handle 406. In this example, the medical device 400 includes a tether 430 extending from a first portion 432 to a second portion 434. The first portion 432 may be coupled to the proximal portion 414 of the hub 412 and the second portion 434 may be coupled to the hub 412 distal of the proximal portion 414. The tether 430 may be elastic, e.g., comprising an elastic material, e.g., to facilitate coupling of the first portion 432 and/or the second portion 434 on the hub 412. In some examples, the first portion 432 of tether 430 may be useful for sealing the proximal opening of the hub 412 prior to insertion of the shaft 401.

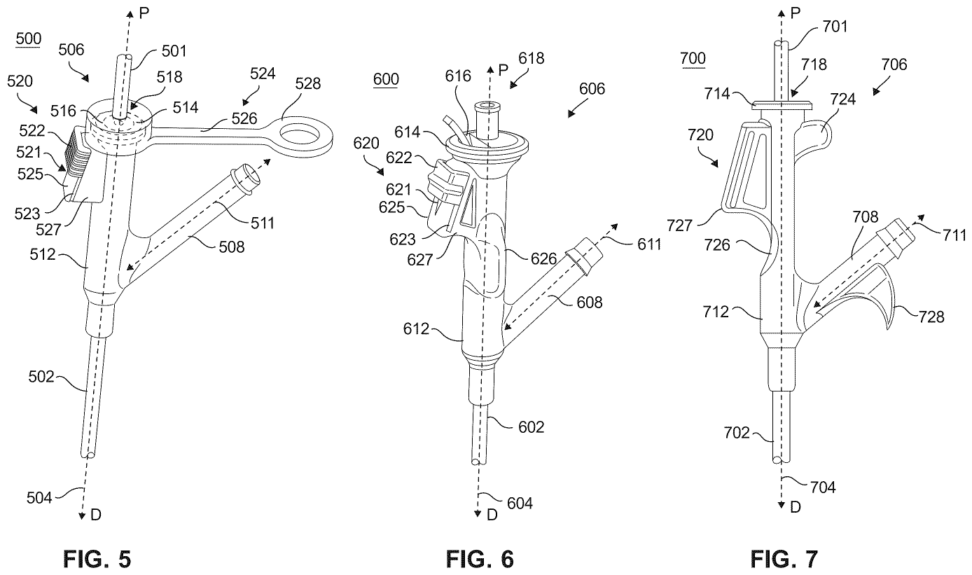

FIG. 5 illustrates another exemplary medical device 500 according to aspects of the disclosure. Unless otherwise specified herein, the medical device 500 may include any of the features of medical devices 100, 200, 300, and/or 400. As shown, the medical device 500 includes a handle 506 and a sheath 502. The handle 506 includes a hub 512 defining a lumen 518 in fluid with a channel defined by the sheath 502. The handle 506 also includes a suction port 508 in fluid communication with the lumen 518 of the hub 512, e.g., the suction port 508 extending along an axis 511 transverse to a longitudinal axis 504 of the hub 512. The hub 512 includes a proximal portion 514 defining a proximal opening 516 capable of receiving a shaft 501 of a medical instrument therein.

The handle 506 includes a ventilation mechanism 520 that includes a housing 527 and an actuator 522 movably coupled the housing 527. For example, the housing 527 may be coupled to, e.g., fixed to, the hub 512, and the actuator 522 may be slidable along the housing 527. Optionally, the housing 527 may include an inclined surface 525. The hub 512 includes one or more apertures 521. The actuator 522 may be slidable along a groove 523 of the housing 527, e.g., the groove 523 defining a range of motion for the actuator 522. The user may slide the actuator 522 relative to the housing 527 to cover and uncover the aperture(s) 521. The user may grasp the handle 506 with the same hand used to move the actuator 522.

In some examples, the ventilation mechanism 520 may include a latch, lock or similar retention mechanism(s) configured to retain a position of the actuator 522 within the groove 523. Optionally, the medical device 500 includes a tether 524 similar to the tether 430 of the medical device 400 (FIGS. 4A-4B). As shown in FIG. 5, the tether 524 may include a first end 526 attachable to the proximal portion 514 of the hub 512 (e.g., to close or seal the opening 516 formed therein), and a second end 528 opposite the first end 526 attachable to the hub 512 (e.g., distal the inclined surface 525 and proximal of the suction port 508 relative to the longitudinal axis 504).

FIG. 6 illustrates another exemplary medical device 600, according to aspects of the present disclosure. Unless otherwise specified herein, the medical device 600 may include any of the features of medical devices 100, 200, 300, 400, and/or 500. As shown, the medical device 600 includes a handle 606 and a sheath 602, the handle 606 including a hub 612 defining a lumen 618 in fluid communication with a channel defined by the sheath 602. The handle 606 also includes a suction port 608 in fluid communication with the lumen 618, e.g., the suction port 608 extending along an axis 611 transverse to a longitudinal axis 604 of the hub 612. The hub 612 may include a proximal portion 614 defining a proximal opening 616 capable of receiving a shaft of a medical instrument therein. The proximal opening 616 may include a seal, e.g., to assist in providing an airtight and/or watertight seal with the shaft received therein.

The handle 606 also includes a ventilation mechanism 620. The ventilation mechanism 620 may include any of the features of the ventilation mechanisms 120, 220, 320, 420, and/or 520 discussed herein. In the example shown in FIG. 6, the ventilation mechanism 620 includes a housing 627 and an actuator 622 movable relative to the housing 627. The housing 627 may be coupled to, e.g., fixed to, the hub 612. Optionally, the housing 627 may include an inclined surface 625. The hub 612 includes an aperture 621 formed therein. The actuator 622 may be slidable along a groove 623 of the housing 627 in a manner similar to medical device 500. As the user slides the actuator 622 relative to the housing 627, the actuator 622 covers and uncovers the aperture 621 to thereby control pressure of the medical device 600. In some examples, the housing 627 and/or hub 612 includes one or more indicators on an outer surface thereof to indicate to the user one or more directions for sliding the actuator 622 and pressure change corresponding to the directions.

In some aspects, the handle 606 may have one or more surface features to assist a user with grasping and/or controlling the medical device 600. These surface features may include ergonomic features that, for example, may increase surface area of the handle 606 graspable by the user. For example, as shown, the handle 606 may include a flattened portion 626, (e.g., adjacent or distal of the ventilation mechanism 620. The flattened portion 626 may be useful, for example, to increase surface area of the handle 606 graspable by the user. Any of the previous and other medical devices herein including medical devices 100, 200, 300, 400, and/or 500 may also include a flattened portion.

FIG. 7 illustrates another exemplary medical device 700, including a handle 706 and a sheath 702. Unless otherwise specified, the medical device 700 may include any of the features of medical devices 100, 200, 300, 400, 500, and/or 600. The handle 706 includes a hub 712 defining a lumen 718 extending therein along a longitudinal axis 704 and in fluid communication with a channel of the sheath 702. The hub 712 includes a proximal portion 714 having a proximal opening capable of receiving a shaft 701 or the like of a medical instrument therein. The handle 706 also includes a suction port 708 configured to be connected to a vacuum source, for example the suction port 708 extending along an axis 711.

The handle 706 also includes a ventilation mechanism 720 that includes a housing 727 and an actuator movable relative to the housing 727. The actuator of ventilation mechanism 720 may be the same as actuators 522 and 622. Optionally, the housing 727 may include an inclined surface. The housing 727 is coupled to, e.g., fixed to, the hub 712. The hub 712 includes one or more apertures formed therethrough that may be covered/uncovered by moving the actuator similarly as described above in connection to medical devices 500 and 600. For example, the actuator of ventilation mechanism 720 may be slidable along a groove of the housing 727.

In some aspects, the handle 706 may include one or more features that support or otherwise facilitate grasping or controlling of the medical device 700 by the user (e.g., for performing the medical procedure). For example, the handle 706 may include a hook 728 extending outwardly from an outer surface of the suction port 708 relative to the axis 711. In the example shown in FIG. 7, the hook 728 has a curved surface oriented in a distally-facing direction (e.g., relative to the longitudinal axis 704) which may be useful, for example, to support one or more fingers for grasping the medical device 700, such as a pinky finger of the user. This may also be useful, for example, to support insertion of the shaft 701 into the lumen 718 and/or removal of the shaft 701 therefrom (e.g., via the proximal opening formed in the proximal portion 714 into/through the lumen 718 defined therein and into/through the channel of the sheath 702).

Additionally or alternatively, the handle 706 may include a tab 724, e.g., positioned along the hub 712. For example, the tab 724 may be proximal to the suction port 708 and distal to the proximal portion 714 of the hub 712. In the example shown, the tab 724 is positioned along the outer surface of the hub 712, adjacent the proximal portion 714 and diametrically opposite the ventilation mechanism 720. The tab 724 may be shaped and sized to contact one or more of the user's fingers while inserting the shaft 701 or the like into the proximal portion 714, such that proximal movement of the handle 706 along the longitudinal axis 704 is inhibited by engagement between the user's fingers and the tab 724.

Further, for example, the handle 706 may include one or more indents shaped and sized to receive one or more fingers of the user's hand to facilitate grasping and controlling the handle 706. In the example shown in FIG. 7, the handle 706 includes an indent 726 along the outer surface of the hub 712, the indent 726 being distal to the ventilation mechanism 720 and proximal the suction port 708. The indent 726 defines a cross-section of the hub 712 having a narrowed cross-section relative to the proximal portion 714. The indent 726 may be useful, for example to facilitate grasping of the medical device 700 by the user. For example, a middle finger of the user may be received in the indent 726 to facilitate grasping and controlling the handle 706.

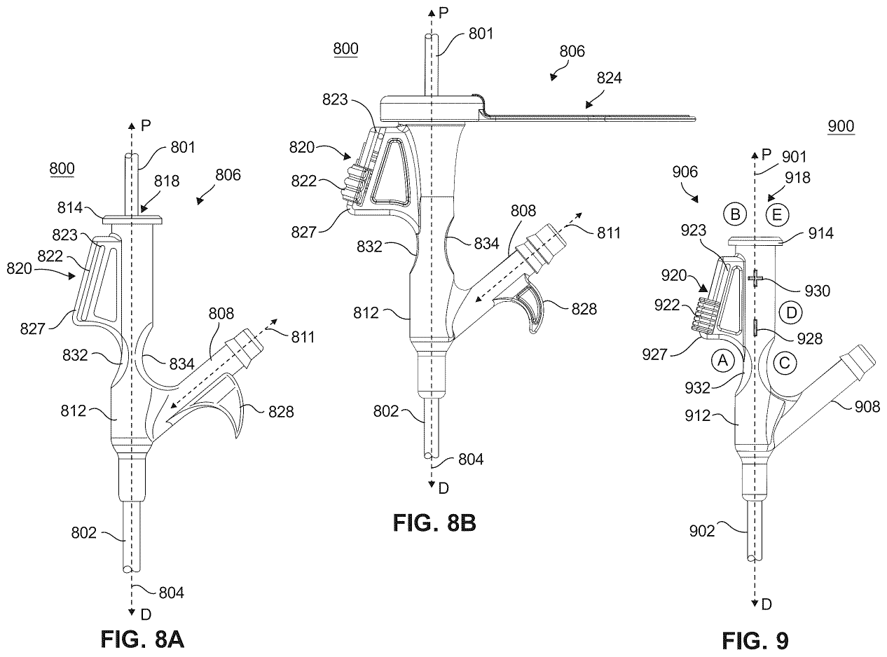

FIGS. 8A-8B illustrate another exemplary medical device 800 including a handle 806 and a sheath 802, according to aspects of the disclosure. The medical device 800 may include any of the features of medical devices 100, 200, 300, 400, 500, 600, and/or 700 unless otherwise specified. As shown, the handle 806 includes a hub 812 defining a lumen 818 extending therein along a longitudinal axis 804 and in fluid communication with a channel of the sheath 802. The hub 812 includes a proximal portion 814 having a proximal opening capable of receiving, for example, a shaft 801 of a medical instrument therein. The handle 806 also includes a suction port 808 configured to be connected to a vacuum source. The handle 806 may also include a ventilation mechanism 820 including a housing 827 and an actuator 822 movable relative to the housing 827, e.g., slidable along a surface of the housing 827. Optionally, the housing 827 may have an inclined surface. The hub 812 includes one or more apertures formed therethrough in fluid communication with the lumen 818. For example, the actuator 822 may be slidable along a groove 823 of the housing 827, such that the user may slide the actuator 822 over the aperture(s) of the hub 812 at least between an open position to uncover/ventilate the aperture(s) and a closed position to cover/seal the aperture(s).

Optionally, the medical device 800 includes a tether 824 similar to the tethers 430 524, of the medical devices 400, 500 discussed above. For example, as shown in FIG. 8B, the tether 824 may have at least one end configured to be coupled with the proximal portion 814 of the hub 812, e.g., to selectively seal/unseal the proximal opening of the hub 812.

The handle 806 may include one or more features that support or otherwise facilitate grasping of the medical device 800 by the user. In some examples, the handle 806 may include a hook 828, e.g., positioned on the suction port 808. In the example shown, the hook 828 has a curved surface oriented in a distally-facing direction (e.g., relative to the longitudinal axis 804). The hook 828 may be useful, for example, to support one or more fingers of the user while grasping the handle 806, such as a pinky finger of the user.

Additionally or alternatively, the handle 806 may include one or more indents shaped and sized to receive one or more fingers of the user to facilitate grasping the handle 806. In the example shown in FIGS. 8A-8B, the handle 806 includes a pair of indents 832, 834 formed along the hub 812 (e.g., distal of the ventilation mechanism 820). For example, the first indent 832 may be positioned on the outer surface diametrically opposed to the second indent 834 relative to longitudinal axis 804, such that the pair of indents 832, 834 collectively define a cross-section along the hub 812 that is having a narrowed cross-section relative to the proximal portion 814 of the hub 812. In some examples, the indents 832, 834 may be useful to facilitate grasping of the medical device 800. For example, a middle finger of the user's hand may be received in the first indent 832 and a ring finger of the same user's hand may be received in the second indent 834 to grasp and control the handle 806.

FIG. 9 illustrates another exemplary medical device 900 including a handle 906 and a sheath 902. Unless otherwise specified, the medical device 900 may include any features of medical devices 100, 200, 300, 400, 500, 600, 700, and/or 800. As shown, the handle 906 includes a suction port 908 configured to be connected to a vacuum source and a hub 912 defining a lumen 918 in fluid communication with the suction portion 908 and a channel defined by the sheath 902. The hub 912 includes a proximal portion 914 having a proximal opening capable of receiving a shaft 901 of a medical instrument therein.

In the example shown, the handle 906 includes a ventilation mechanism 920 that includes a housing 927 and an actuator 922. The housing 927 may be coupled to, e.g., fixed to, the hub 912 distal to the proximal opening. The actuator 922 may be slidable relative to the housing 927, e.g., within a groove 923 of the housing 927. Optionally, the housing 927 may have an inclined surface. The hub 912 includes one or more apertures formed therethrough, such that the aperture(s) are directly or indirectly in fluid communication with the lumen 918 of the hub 912. As a user slides the actuator 922 over the housing 927, the actuator 922 gradually covers or uncovers the aperture(s) of the hub 912 to gradually decrease or increase pressure of the medical device 900. For example, the user may control pressure via the ventilation mechanism 920 with one hand, and use the other hand to insert or remove the shaft 901 via the proximal opening of the hub 912.

In some examples, the handle 906 includes one or more visual indicators corresponding to one or more directions for moving the actuator 922. For example, the handle 906 may include a first indicator 928 and a second indicator 930 to show corresponding first and second directions for sliding the actuator 922. In the example shown, the first indicator 928 is the subtraction symbol (−) corresponding with a decrease in pressure, and the second indicator 930 is the addition symbol (+) corresponding with an increase in pressure therethrough.

In some aspects, the medical device 900 may be ergonomically configured to accommodate the user's grip. In the example shown in FIG. 9, several labels (A-E) are provided for illustrative purposes to show potential finger placement for a user's hand. Label A shows an exemplary position for the index finger of the user's hand (e.g., at an indent 932 formed on the outer surface of the hub 912), label B shows an exemplary position for the thumb, label C shows an exemplary position for the middle finger, label D shows an exemplary position for the ring finger, and label E shows an exemplary position for the pinky finger.

While principles of this disclosure are described herein with reference to illustrative examples, it should be understood that the disclosure is not limited thereto. Those having ordinary skill in the art and access to the teachings provided herein will recognize additional modifications, embodiments, and substitution of equivalents all fall within the scope of the features described herein. Accordingly, the claimed features are not to be considered as limited by the foregoing description.

What is claimed is:

1. A medical device, comprising:
a handle including:
a hub defining a lumen and a proximal opening configured to receive a shaft of a medical instrument;
a suction port in fluid communication with the lumen and configured to be connected to a vacuum source; and
a ventilation mechanism including a housing coupled to the hub; and
a sheath coupled to the handle and defining a channel in fluid communication with the suction port;
wherein the hub or the suction port includes at least one aperture, and the ventilation mechanism includes an actuator is movable relative to the hub to cover and uncover the at least one aperture.

2. The medical device of claim 1, wherein an outer surface of the housing is the actuator, the housing being movable relative to the hub.

3. The medical device of claim 1, wherein the suction port includes the at least one aperture, and wherein the housing is pivotably coupled to the hub.

4. The medical device of claim 3, wherein the ventilation mechanism includes a spring within the housing, the spring being configured to bias the housing toward the suction port.

5. The medical device of claim 1, wherein the at least one aperture has a variable cross-sectional profile.

6. The medical device of claim 1, wherein the hub includes the at least one aperture.

7. The medical device of claim 6, wherein the housing is fixed to the hub and the actuator is movably coupled to the housing.

8. The medical device of claim 7, wherein the actuator is slidable along a surface of the housing.

9. The medical device of claim 6, wherein the housing surrounds a portion of the hub.

10. The medical device of claim 9, wherein the housing is slidable relative to the hub along an axis perpendicular to a longitudinal axis of the hub.

11. The medical device of claim 9, wherein the housing is rotatable relative to the hub about an axis perpendicular to a longitudinal axis of the hub.

12. The medical device of claim 9, wherein the housing is rotatable relative to the hub about a longitudinal axis of the hub.

13. The medical device of claim 6, wherein the at least one aperture includes a plurality of apertures that includes a first aperture adjacent to a second aperture at a same axial position along a length of the hub.

14. The medical device of claim 6, wherein the at least one aperture includes a plurality of apertures that includes a first aperture proximal to a second aperture along a length of the hub.

15. The medical device of claim 1, wherein the hub includes a tab, a hook, or a flattened portion distal to the housing and configured to be gripped by a hand of a user.

16. A medical device, comprising:
a handle including:
a hub defining a lumen and a proximal opening configured to receive a shaft of a medical instrument, wherein the hub includes at least one aperture;
a suction port in fluid communication with the lumen of the hub and configured to be connected to a vacuum source; and
a ventilation mechanism including a housing coupled to the hub; and
a sheath coupled to the handle and defining a channel in fluid communication with the lumen of the hub;
wherein the ventilation mechanism includes an actuator movable relative to the hub to cover and uncover the at least one aperture, the actuator being an outer surface of the housing or coupled to and slidable relative to the housing.

17. The medical device of claim 16, wherein the housing surrounds a portion of the hub, the housing being slidable or rotatable relative to the hub.

18. The medical device of claim 16, wherein the housing is fixed to the hub and the actuator is slidable along a surface of the housing.

19. A medical device, comprising:
a handle including:
a hub defining a lumen and a proximal opening configured to receive a shaft of a medical instrument, wherein the hub includes at least one aperture;
a suction port in fluid communication with the lumen and configured to be connected to a vacuum source; and
a ventilation mechanism including a housing coupled the hub proximate the at least one aperture, wherein the housing is slidable relative to the hub to cover and uncover the at least one aperture; and
a sheath coupled to the handle and defining a channel in fluid communication with the lumen of the hub.

20. The medical device of claim 19, wherein the hub includes a tab, a hook, or a flattened portion distal to the housing and configured to be gripped by a hand of a user.
